(19) United States
(12) Patent Application Publication
(10) Pub. No.: US 2026/0306694 A1
(43) Pub. Date: Oct. 8, 2026

(54) CHANNELED AIR FRESHENER WITH MULTIPLE RELEASE MODES

(71) Applicant: Energizer Auto, Inc., St. Louis, MO (US)

(72) Inventors: Elin Lee LeClaire, Sheffield Village, OH (US); Anthony D'Alessandro, North Olmsted, OH (US)

(21) Appl. No.: 19/640,970

(22) Filed: Apr. 7, 2026

Related U.S. Application Data
(60) Provisional application No. 63/785,279, filed on Apr. 8, 2025.

(51) Int. Cl. A61L 9/12; A61L 9/04

(52) U.S. Cl. CPC A61L 9/127; A61L 9/042; A61L 2209/133; A61L 2209/134; A61L 2209/15

(57) ABSTRACT

An air freshener includes a scented substrate encased on one side by a fibrous plate and on other sides by a housing. The substrate is loaded with fragrance oil, which the plate wicks from the substrate and diffuses into the environment. Openings in the plate align with passageways in the substrate to form channels for airflow. The airflow may rise through the channels, extract fragrance from the plate and substrate, and exit the housing through vents positioned opposite the plate and substantially orthogonal to the channels. The channeled air freshener may be clipped to a vehicle sun visor to diffuse fragrance into the environment in multiple modes.

### CROSS REFERENCE TO RELATED APPLICATION

This application claims priority to U.S. Provisional Application Ser. No. 63/785,279 filed on Apr. 8, 2025, and entitled, “CHANNELED AIR FRESHENER WITH MULTIPLE RELEASE MODES, the contents of which are incorporated herein by reference.

### TECHNICAL FIELD

The present disclosure relates generally to a scented substrate configured to diffuse fragrance to the environment as an air freshener. More specifically, the present disclosure relates to a scented substrate and fibrous panel containing channels for directing airflow and having multiple modes for diffusing fragrance.

### BACKGROUND

In general, air convection and heat can help dissipate fragrance oils from an air freshener into an ambient space to provide an aroma. In vehicles, air fresheners are often positioned to use airflow from the ventilation system to dissipate the fragrance within the vehicle interior. For instance, the device may be placed within louvers of an air vent of the ventilation system, using forced airflow and possibly heat through the vents to disperse the fragrance. Placement of an air freshener on or over vents in the vehicle can interfere with airflow within the vehicle, making the ventilation system less effective at heating or cooling the vehicle or in directing a flow of air to occupants.

For implementations away from forced air, challenges exist with diffusing sufficient fragrance. In some approaches, the surface area of a fragrant substrate is increased by forming voids or indentations within the substrate. The larger surface area can increase the exposure of the fragrance oils to natural airflow and subsequent diffusion. In many situations, however, the creation of voids or cavities within a fragrance substate can cause air surrounding the substrate to become entrapped, reducing the effectiveness of fragrance release. Entrapment of the air by the substrate may not be overcome in the absence of forced air when heat differences around the device, such as within the interior of a vehicle, cause natural airflow. The air entrapment may be enhanced when the fragrant substrate is enclosed within a protective housing.

Examples of the present disclosure are directed to overcoming deficiencies of such systems.

### SUMMARY

In an aspect of the present disclosure, an air freshener includes a fragrant body containing fragrance oil where the fragrant body has at least an inner portion and an outer surface and the outer surface includes a fibrous material. The air freshener further includes channels of open space passing through the fragrant body from the inner surface to the outer surface, and a housing configured to encase the inner portion of the fragrant body. The housing and the outer surface of the fragrant body form an exterior of the air freshener.

In another aspect of the present disclosure, an air freshener includes a scented substrate loaded with an oil-based fragrance, passageways for airflow formed through the scented substrate, a housing configured to encase the scented substrate, and a gap between the scented substrate and the housing. The housing includes one or more vents positioned orthogonal to the passageways, and the gap provides a path for the airflow from one or more of the passageways to the one or more vents.

In yet another aspect of the present disclosure, a diffuser includes a plate of fibrous material, openings for airflow formed through the fibrous material, a scented substrate loaded with an oil-based fragrance, and passageways for the airflow formed through the scented substrate. The scented substrate is in contact with the plate of fibrous material, and the openings are aligned with the passageways to form channels for the airflow through the diffuser.

### BRIEF DESCRIPTION OF DRAWINGS

FIG. 1 is an isometric view of an air freshener installed on a visor of a vehicle in accordance with an example of the present disclosure.

FIG. 2 is an exploded view of the air freshener of FIG. 1 in accordance with an example of the present disclosure.

FIG. 3 is an isometric view of the air freshener of FIG. 1 in a state of assembly prior to attachment of a housing in accordance with an example of the present disclosure.

FIG. 4 is cutaway view of the air freshener of FIG. 1 in accordance with an example of the present disclosure.

FIG. 5 is a line graphs showing a comparison of perceived scent strength over time between a partial product without a scented substrate and a whole product with a scented substrate in accordance with an example of the present disclosure.

### DETAILED DESCRIPTION

Wherever possible, the same reference numbers will be used throughout the drawings to refer to same or like parts.

In accordance with the principles discussed in the present disclosure, which are detailed in the following examples, an air freshener includes a scented substrate encased on one side by a fibrous plate and on other sides by a housing. The substrate is loaded with fragrance oil, which the plate wicks or absorbs from the substrate and then diffuses into the environment. Openings in the plate may be aligned with passageways in the substrate to form channels for airflow. The airflow may rise through the channels, extract fragrance from the plate and substrate, and exit the housing through vents positioned opposite the plate and substantially orthogonal to the channels. The channeled air freshener may be clipped to a vehicle sun visor to diffuse fragrance into the environment around the freshener in multiple modes.

Additionally, a hybrid air freshener consistent with the principles of the present disclosure may be configured to diffuse fragrance oils into an ambient environment through multiple modes while protecting against unnecessary contact of the fragrance oils by a user. As discussed in various examples below, a housing and a wood plate may form an exterior of the device, encasing a substrate loaded with fragrance oil. This structure can protect a user's skin from possible irritation from fragrance oils when handling the air freshener and protect surrounding surfaces, such as a vehicle interior, from contacting the oils. The following describes several examples for carrying out the principles of this disclosure.

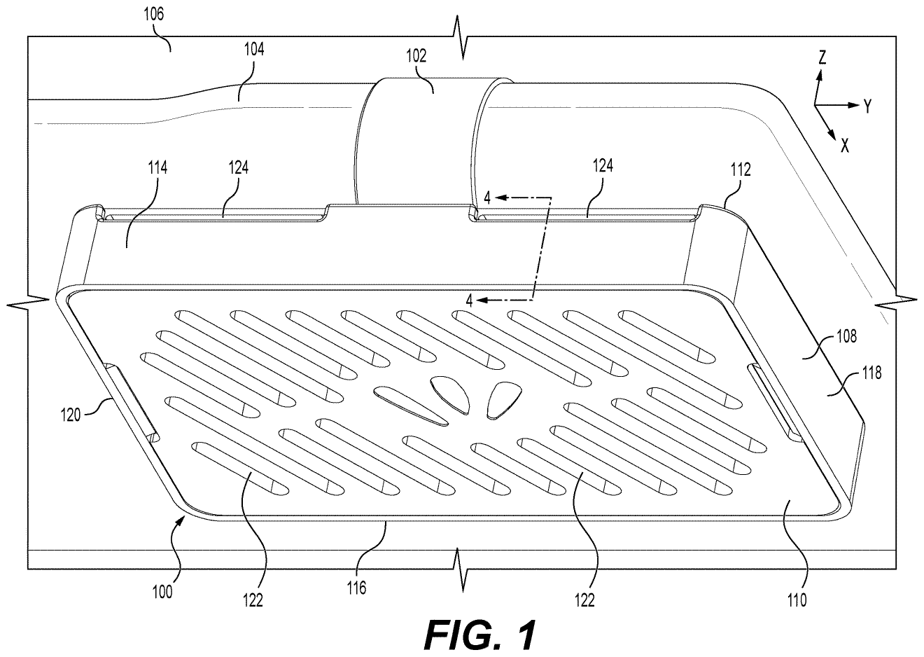

FIGS. 1-4 illustrate different views and components of an exemplary air freshener 100 discussed in the present disclosure. FIG. 1 illustrates an isometric view of the front, bottom, and right side of the air freshener within an XYZ coordinate system as one example. As generally embodied in FIG. 1, air freshener 100 has the overall form resembling a rectangular prism extending within the X-Y plane. In this example, the dimension of air freshener 100 along the Y axis is longer than its dimension along the X axis, which is longer than its dimension along the Z axis. In some examples, air freshener 100 is about 8 cm long (in the Y direction), about 5 cm wide (in the X direction), and about 1 cm high (in the Z direction). This shape for air freshener 100 is representative only. The air freshener 100 may be of any size or shape desired for a particular implementation and could alternatively resemble a cuboid, an oval, a sphere, or any other form.

As shown in FIG. 1, the exterior of air freshener 100 may include a housing 108 to which clip 102 is attached and a bottom plate 110. Housing 108 has a top 112, a front 114, a rear 116, a first side 118, and a second side 120. Thus, in the example of FIG. 1, five of six sides of the rectangular prism of air freshener 100 are formed by housing 108. The sixth side is formed by bottom plate 110. The bottom plate 110 includes openings 122, and housing 108 includes vents 124, which together define paths for airflow in one mode for dispersing fragrance in a manner discussed in more detail below.

Typically, air freshener 100 is installed within the cab or interior of a vehicle and passively disperses fragrance within that region. In the example of FIG. 1, air freshener 100 has a shape resembling a garage-door opener and includes a clip 102 for slidably attaching air freshener 100 to a visor 104. Most commonly, visor 104 is installed above the windshield and, when in an up position, is substantially parallel or adjacent to a ceiling 106 of the vehicle. When in a down position (not shown), visor 104 can be substantially parallel to the windshield for blocking glare from the sun or other lights coming through the windshield. Therefore, FIG. 1 generally illustrates the view of 100 from the perspective of a driver and/or passenger looking upward at visor 104 toward the bottom of the installed air freshener.

In most situations, air freshener 100 does not need forced air, such as air forced from vents in an HVAC system of a vehicle, to dispense fragrance. While forced air may enhance dispersion of fragrance, the structure of air freshener 100 as discussed below provides mechanisms for enhancing the dispersion of fragrance without requiring forced air convection. Accordingly, air freshener 100 may be placed not only on visor 104 of a vehicle, but also in other locations needing a fragrant or deodorizing scent, such as other locations within the interior of a vehicle (e.g., a rear seat pocket, a car door pocket, a grab handle, a seat belt), a room or office of a building, or any other closed environment with or without forced air.

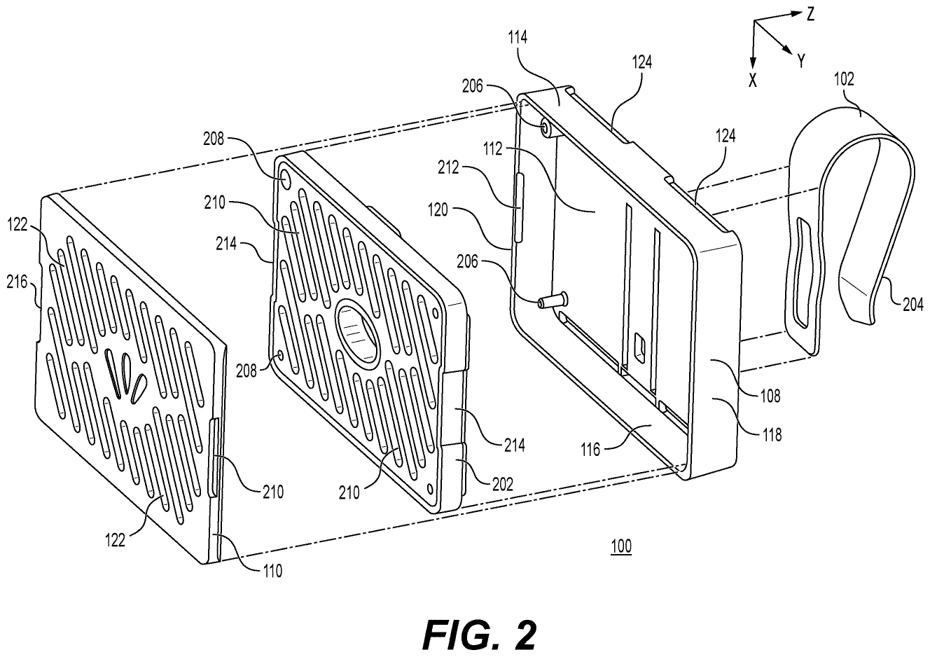

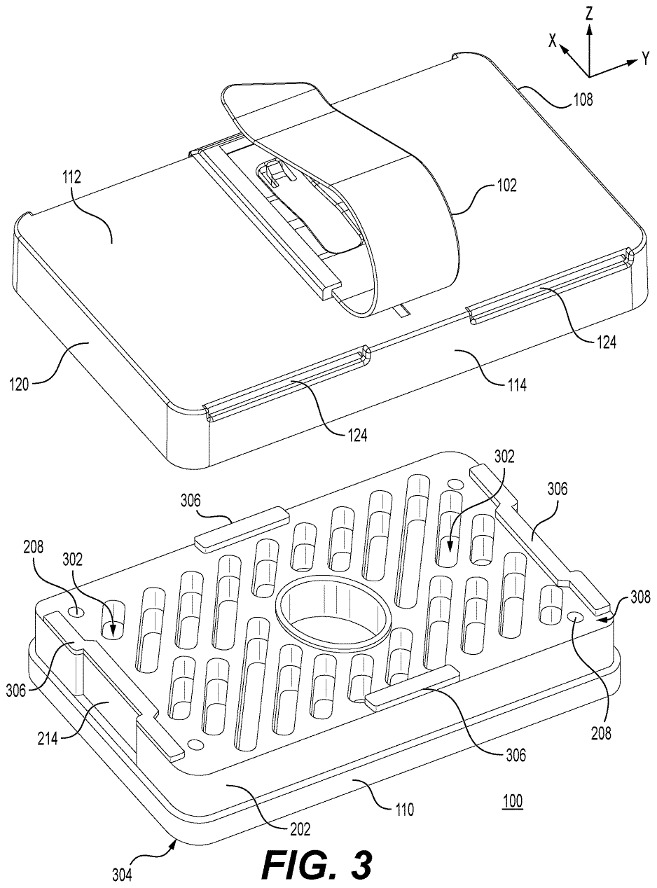

While FIG. 1 illustrates the exterior of air freshener 100 as installed on clip 102 of a vehicle, FIG. 2 is an exploded view of air freshener 100 showing its four primary components. From right to left in FIG. 2, the disassembled air freshener 100 includes clip 102, housing 108, scented substrate 202, and bottom plate 110. Clip 102 may include at least one leg 204 made of a curved, resilient material arranged to exert a force against visor 104 or a similar mounting structure in a known manner when slid into place. In some examples, leg 204 is plastic, stainless steel, or aluminum and may include modifications to its surface area (such as with rubber or silicone or similar material) sufficient to enhance a gripping action when placed around a mounting structure or plane, such as visor 104. The curved, or U-shaped, structure for leg 204 shown in the figures is exemplary, and any other shape or clamping function for clip 102 sufficient to attach air freshener 100 to a structure would satisfy the objectives of clip 102 for the present disclosure. Clip 102 may be attached to top 112 of housing 108 by any means suitable for the implementation, such as by a fastener, an adhesive, an interlocking arrangement between clip 102 and top 112 (see FIG. 3), or any other techniques.

As explained above for FIG. 1, in the illustrated examples, housing 108 may resemble a rectangular box without its lid. In some examples, housing 108 is made of a thermoplastic polymer, such as acrylonitrile butadiene styrene (ABS). The housing may be an oil-resistant plastic, such as polyoxymethylene (POM) or polypropylene, with or without additional parts of the same or different plastic, (e.g., ABS, ceramics, or glass) or an enclosing permeable material (e.g., a woven or nonwoven fabric, paper-based, vented glass, or ceramic or perlite material). When oil-resistant, the material of housing 108 may minimize direct contact between any scented substrate within air freshener 100 and a surface of the vehicle or the hands of a user. Alternatively, housing 108 may be formed from other lightweight, strong, and durable materials such as different types of plastics or metals, such as aluminum.

When air freshener 100 is assembled, housing 108 and bottom plate 110 encase scented substrate 202. Scented substrate 202 is any solid, moldable material capable of holding or retaining fragrance oil. In some examples, scented substrate 202 comprises a fragrance oil mixed and compounded within a polymer. The polymer may be any moldable plastic commonly used in the field, such as polyvinylchloride (PVC), ethylene-vinyl acetate (EVA), polyurethane (PU), polyethylene (PE), or similar materials. Other solid moldable materials, such as ceramics or gypsum, could be substituted as the substrate.

As shown, scented substrate 202 has a substantially planar shape, typically formed through molding, with dimensions sufficient to fit securely within a cavity of housing 108 formed within front 114, rear 116, first side 118, and second side 120. While shown as rectangular, scented substrate 202 may have any other desired shape, such as triangular, oval, airfoil, teardrop, polygonal, etc., that allows air freshener 100 to function for the purposes described in this disclosure. Scented substrate 202 may be retained within 108 through the insertion of one or more pins 206 mounted on top 112 of housing 108 into one or more corresponding registration holes 208 in scented substrate 202.

Functioning as a substrate, scented substrate 202 contains fragrance oils that can be emitted or dispersed from scented substrate 202 to produce a desired scent or fragrance to freshen the ambient air (e.g., provide a desired scent for the air or remove or mask an unwanted or unpleasant odor in the air). The scent or fragrance can be any of a variety of types, including, without limitation, fruit scents, food scents, floral scents, water or ocean themed scents, and natural scents among others.

As illustrated in FIG. 2, scented substrate 202 includes one or more passageways 210 that pass through a thickness of scented substrate 202, e.g., through the X-Y plane formed by scented substrate 202 in a direction parallel to the Z axis. The passageways 210 can be of any shape, size, or quantity and serve as cavities or orifices for the passage of air through scented substrate 202. In the example of FIG. 2, the twenty-two illustrated passageways 210 are generally oblong in shape, resembling a rectangle with rounded ends and having various lengths. Other shapes and quantities for passageways 210 are within the scope of the present disclosure.

At the far left of FIG. 2, bottom plate 110 provides an outer wall completing the rectangular box or prism of housing 108. Bottom plate 110 is generally planar in shape and is a porous and fibrous material, such as a lignin-based substance. While described as a plate for descriptive purposes, bottom plate 110 is a fibrous material that need not be configured as a plate or planar structure and may take any shape or form appropriate for the implementation. In one example, bottom plate 110 may be wood. Beechwood provides one option for a porous and fibrous material that also provides a generally pleasing appearance on the exterior of bottom plate 110, although any cut wood may suffice. Depending on a desired appearance, other cellulose-based or wood-based materials having a porous and fibrous consistency, such as pressed wood products, bamboos, molded pulp products, papers and paperboards, woven or nonwoven fibers or fabrics, and cellulose sponge materials could serve as the material for bottom plate 110.

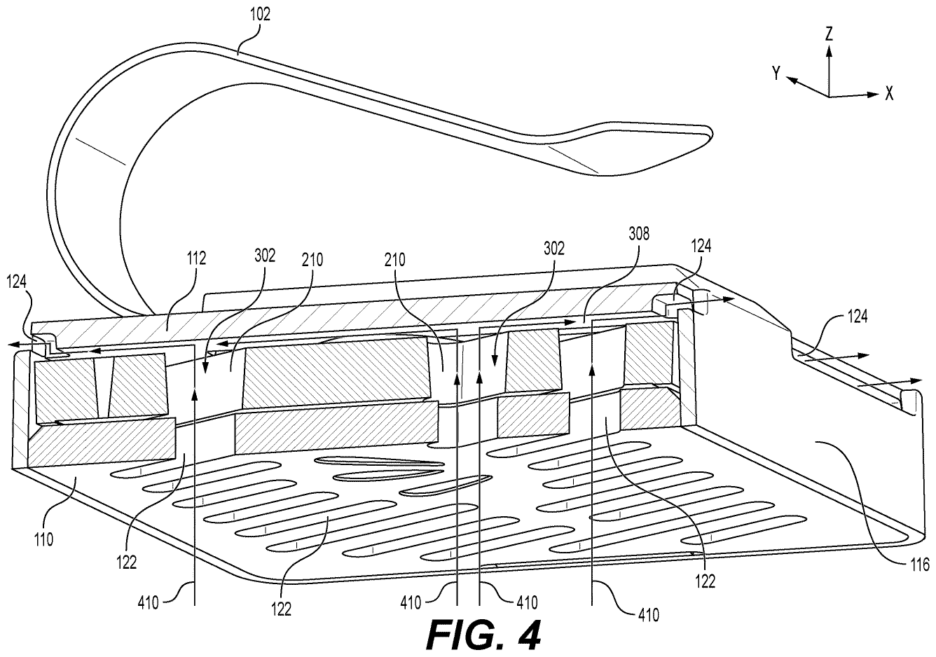

Similarly to scented substrate 202, the one or more openings 122 pass through a thickness of bottom plate 110, e.g., through the X-Y plane formed by bottom plate 110 in a direction parallel to the Z axis. The openings 122 can be of any shape, size, or quantity and serve as cavities or orifices for the passage of air through bottom plate 110. In the example of FIG. 2, the twenty-two illustrated openings 122 are generally the same size and shape as passageways 210, being oblong and having various lengths. As shown, when the same size and shape, openings 122 and passageways 210 may be aligned in registration when air freshener 100 is assembled, forming a plurality of channels 302 (see FIGS. 3 and 4) as direct paths for air to pass within and through the device. In other options, the size and shape of openings 122 may be different from passageways 210 and may be other than oblong. When different in size and shape, openings 122 and passageways 210 may form a plurality of channels that are indirect paths for air to pass within the device. Other shapes and quantities for passageways 210 are within the scope of the present disclosure.

Bottom plate 110 may be mounted over and in contact with scented substrate 202, being secured to housing 108 to form a complete and tight package for air freshener 100. In the example of FIG. 2, attachment of bottom plate 110 to housing 108 occurs via ridges 212, or lips, on an edge of first side 118 and second side 120 of housing 108. Indentations 214 within scented substrate 202 allow ridges 212 to pass over that substrate and to snap into corresponding recesses 216 on bottom plate 110 when assembled. Other approaches to securing bottom plate 110 and scented substrate 202 in place within housing 108 will be apparent to those of ordinary skill in the field, including the use of fasteners, adhesives, or other interlocking arrangements.

FIG. 3 illustrates a perspective of air freshener 100 prior to housing 108 being attached. In this example, the planar surfaces of bottom plate 110 and scented substrate 202 (i.e., the surfaces within the X-Y plane in the figures) may be initially arranged in contact with each other as shown in FIG. 3 to form, in combination, a fragrant body 304. Bottom plate 110 and scented substrate 202 may be positioned together so that openings 122 in bottom plate 110 align with passageways 210 in scented substrate 202, creating channels 302 as direct paths of open space through the combined structure. The contact between bottom plate 110 and scented substrate 202 may be secured using an adhesive at various locations, a fastener, or a mechanical interlocking arrangement, as desired.

When bottom plate 110 and scented substrate 202 remain in contact over time as the fragrant body 304, fragrant oils dispersed or diffused from the substrate will be absorbed by bottom plate 110. In particular, molecules of the fragrant oils may be released as vapor from scented substrate 202 and, with bottom plate 110 being porous and in contact with scented substrate 202, some of those molecules will migrate into the fibrous structure of bottom plate 110. Bottom plate 110 will, over time, become charged with fragrant oils originally loaded in scented substrate 202. As a result, both bottom plate 110 and scented substrate 202 can function together as sources of fragrance from fragrant body 304.

In completing the structure, housing 108 is fitted over fragrant body 304 as a cover, and pins 206 in housing 108 are aligned with registration holes 208 in scented substrate 202. Ridges 212 (see FIG. 2) will pass through indentations 214 and snap into recesses 216 on bottom plate 110, securing fragrant body 304 into housing 108 and forming air freshener 100 in condition for use. In some examples, the surface of scented substrate 202 facing housing 108 includes one or more spacers 306 formed of any suitable material. When housing 108 is installed over fragrant body 304, spacers 306 will impede the surface of fragrant body 304 from contacting top 112 of housing 108, offsetting scented substrate 202 from top 112 by a gap 308. Air freshener 100 may then be installed in any desired location to diffuse the fragrance.

To help induce wicking and to prepare bottom plate 110 as a source of fragrance when air freshener 100 is initially installed, fragrant body 304 may be packaged in a sealed environment at the time of manufacturing, in some examples, with or without housing 108. For instance, the assembly of fragrant body 304 and housing 108 may be sealed in a blister pack or similar arrangement to prevent scented substrate 202 from dispersing fragrance to the ambient environment, while enabling wicking of molecules of fragrant oil from scented substrate 202 into bottom plate 110. After removing the assembly from the package, a user may install clip 102 as desired prior to installation, or clip 102 may also be pre-assembled with the other components. Other combinations of shipment are possible to contain fragrant body 304 in a sealed environment, such as fragrant body 304 being packaged alone or one or more other components of air freshener 100 also being packaged in the sealed environment, whether assembled or apart.

In accordance with the principles of the present disclosure, hybrid air freshener 100 therefore has a first mode of dispersing fragrance based on diffusion of vapor from bottom plate 110. Due to the packaging of fragrant body 304 in a sealed environment, such as a blister pack, bottom plate 110 will have wicked fragrant oils from scented substrate 202 by the time a user opens the packaging. As a result, bottom plate 110 will be able to diffuse the fragrance immediately after the package is opened and air freshener 100 is installed. Due to its porosity compared with scented substrate 202, which enables its wicking, bottom plate 110 may provide a higher rate of diffusion for the fragrant oils compared with scented substrate 202. As a result, in some examples, bottom plate 110 will enhance an initial release of fragrance to the environment after the package is opened compared with scented substrate 202 alone. While diffusing vapor of the fragrant oils over time, bottom plate 110 will also continue to wick oil molecules from scented substrate 202 due to the substantially larger oil content within scented substrate 202 and to release those molecules as vapor into the environment, such as into the interior of a vehicle.

Moreover, because the fragrant oils are loaded within scented substrate 202 and not stored as a reservoir of liquid, and because the diffusion into the porous fibers of bottom plate 110 occurs as a gas transfer, air freshener 100 does not expose fragrant oils at the exterior of the device. Accordingly, air freshener 100 can minimize direct contact between any fragrant oils and a surface of the vehicle or the hands of a user. For instance, when air freshener 100 is installed on a visor 104 of a vehicle, as shown in FIG. 1, a risk of fragrant oils contacting the user's hands when the device is installed, contacting the visor during operation, or dripping from the device over time are minimized.

In accordance with the principles of the present disclosure, hybrid air freshener 100 includes at least a second mode of dispersing fragrance based on diffusion of vapor within channels 302. FIG. 4 is a sectional view of air freshener 100 with a perspective from the right side of the device in FIG. 1 when sectioned along the cut lines of FIG. 1 (i.e., cut along the X-Z plane near the middle of the device). As shown in FIG. 4, openings 122 in bottom plate 110 and passageways 210 in scented substrate 202 form channels 302 passing in the Z direction. Channels 302 provide open space between ambient atmosphere and gap 308 adjacent top 112. Gap 308, which extends across the facing surfaces of scented substrate 202 and top 112 generally orthogonal to channels 302, likewise provides open space for air to pass to vents 124, which are open to the atmosphere.

In some examples, vents 124 are cavities or separations positioned at the top edge of housing 108. In some examples, vents 124 are located at or near one or more edges of 112. In other examples, vents 124 are located at or near a top edge of one or more of front 114, rear 116, first side 118, and second side 120. In the example in the figures, vents 124 are thin rectangular-shaped openings formed at the intersection of top 112 and front 114 and at the intersection of top 112 and rear 116. The size, shape, and placement of vents 124 are not limited to these examples. While shown as being substantially rectangular, vents 124 may be square, circular, triangular, or any other shape and may occur at any position within the upper portion of air freshener 100 proximate to top 112 as desired without departing from the spirit of this disclosure.

Accordingly, as shown in FIG. 4, airflow 410 may pass through air freshener 100, helping to spread vapor of the fragrant oils into the environment. As shown, airflow 410 may enter one or more of openings 122, pass through corresponding channels 302 into gap 308, and then exit air freshener 100 via either or both of vents 124. Conversely, while not illustrated, airflow 410 may travel the opposite direction, namely, entering vents 124 and passing through gap 308 to channels 302 and exiting the device through openings 122. The plurality of channels 302 helps increase the diffusion of fragrance at least by increasing the surface area of scented substrate 202 for the release of fragrance and providing a pathway for heated air to rise passively into openings 122 and to exit through vents 124. As a result, airflow 410 does not get trapped within the device and can be released with the fragrance into the environment. It will be appreciated that other quantities, shapes, and locations of channels 302 will impact a desired scent intensity dispersed into the environment and duration of scented substrate 202. For instance, although generally indicated as being open paths, channels 302 could be adjustable or closeable to help balance a desired scent intensity and duration for the air freshener. Further, both scented substrate 202 and openings 122 may contribute to the diffusion of scent by airflow 410 since both structures contain molecules of the fragrant oil.

Other features of air freshener 100 may enhance the aroma for a user when the device is located on a visor 104 as in FIG. 1. For example, when air freshener 100 is placed in visor 104 near ceiling 106 of a vehicle with bottom plate 110 facing downward, rising heat will tend to draw airflow 410 upwardly into openings 122 and increase scent dissipation. Fragrance dispersion may also be enhanced by heat radiation from sunlight passing through the windshield and striking rear 116 of air freshener 100. The airflow 410 will rise through channels 302, travel within gap 308 along top 112, and exit housing 108 at either or both of vents 124. The position of vents 124 substantially orthogonal to channels 302 allows the vapor to leave housing 108 without being blocked by visor 104 or other mounting structure, without requiring forced air to cause the vapor to reverse course from gap 308 and be pushed out through openings in having to reverse course within openings 122. Further, forced air from the defroster of the vehicle's ventilation system may be used—purposedly or incidentally—to help dissipate the fragrance oils by convection.

The placement of vents 124 can also improve scent dispersion. For instance, the location of one of vents 124 adjacent front 114 will cause the exit of scent to be directly above and facing the driver or passenger when air freshener 100 is clipped on visor 104. Therefore, the vent near front 114 can help send the aroma more directly to that person. Also, when visor 104 is rotated down to block glare through the windshield, the resulting upward location of the other vent at the intersection of rear 116 and top 112 will also help disperse the vapor as it rises upwardly as with a chimney.

To confirm the improvements provided by an air freshener with multiple release modes according to the present disclosure, various tests were performed. In one test, four products were provided to a panel of twenty-two individuals. Those four products conformed to air freshener 100 as depicted in FIGS. 1-4 and, respectively, were: (1) a whole product loaded with a first fragrance; (2) a partial product loaded with the first fragrance after scented substrate 202 was removed; (3) a whole product loaded with a second fragrance; and (4) a partial product loaded with the second fragrance after scented substrate 202 was removed. Thus, the source of fragrance for whole products (1) and (3) was both a substrate charged with fragrance oil, i.e., scented substrate 202, and a fibrous material, i.e., bottom plate 110, while the source of fragrance for partial products (2) and (4) was only the fibrous material. The fibrous material for the tests was beechwood. The panelists rated the fragrance strength for each of the four products over 4.5 hours.

The detected difference in fragrance strength between products (1) and (2) and between products (3) and (4) was calculated to determine the relative strength provided by the fibrous material. Specifically, for each fragrance, the percentage of fragrance strength of the whole product caused by the fibrous material (i.e., bottom plate 110) was determined according to the equation {1−[(partial product rating-whole product rating)/whole product rating)]}*100. The results showed that the contribution of fragrance release by bottom plate 110 was 85% for the first fragrance and 93% for the second fragrance. The pooled value was 89%. These tests confirm that the fibrous material within bottom plate 110 absorbs fragrance from scented substrate 202 and re-releases it, which enhances the user experience particularly at the time the packaging for the product is initially opened.

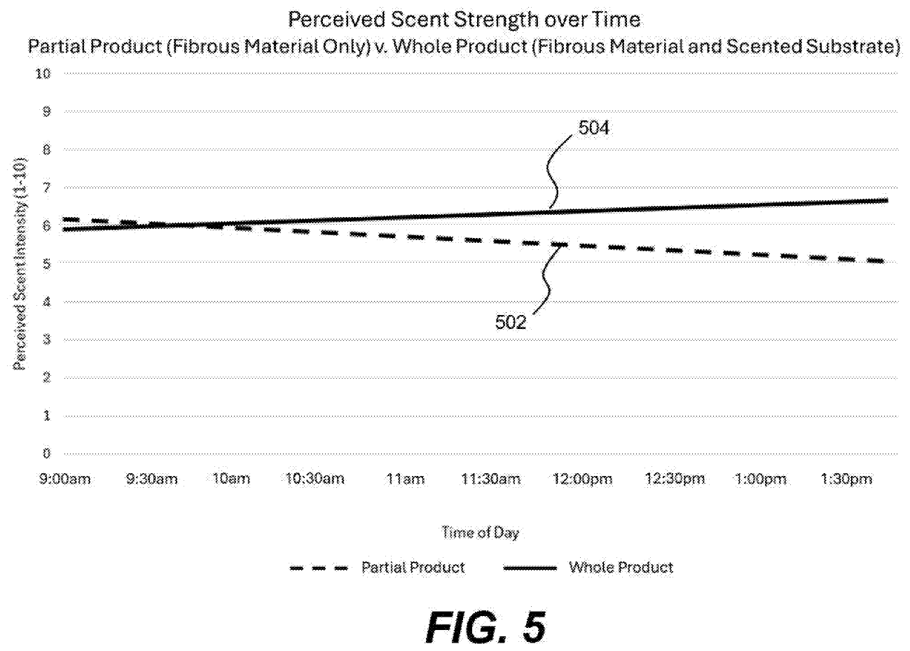

FIG. 5 is a line graph showing a comparison of perceived scent strength on a scale of 1-10 as detected by the panelists over the 4.5-hour rating period. As shown, the average perceived scent intensity for a partial product having the scented substrate removed, referenced as result 502, decreased over time. Result 502 indicates that, after initial loading of fragrance, the fibrous material no longer had the ability to wick additional fragrance molecules from the scented substrate since the substrate had been removed. In contrast, the average perceived scent intensity of the whole product increased over time, referenced as result 504. Result 504 indicates that the scented substrate can wick fragrance into the bottom plate and add fragrance to the atmosphere over time. Further, the results suggest that fragrance released into the air by the substrate and wicked into the fibrous material of the bottom plate diffuse into the air at different rates. Both mechanisms for diffusing fragrance can operate to enhance the user experience.

Accordingly, a hybrid air freshener consistent with the principles of this disclosure may provide a diverse arrangement for delivering fragrance to an interior space, such as the cab of a vehicle. In one mode, air freshener 100 can diffuse vapor of fragrance oil from bottom plate 110 on the exterior of the device. Placed in contact with, and possibly attached to, scented substrate 202, a fibrous material of bottom plate 110, such as beechwood, can wick vapor of the fragrance oil from scented substrate 202 and then diffuse the vapor into the environment. When air freshener 100 is positioned with clip 102 on a visor 104 of a vehicle, bottom plate 110 will face downward in proximity to a driver or passenger and help deodorize the air. With scented substrate 202 contained within the interior of air freshener 100 by housing 108 and bottom plate 110, the fragrance oil embedded within the substrate cannot easily escape to the exterior of the device, limiting the risk of contacting the interior of the vehicle, contacting a person's hands, or accumulating on the exterior of the device. By packaging bottom plate 110 and scented substrate 202 together in a sealed covering prior to use, molecules of the fragrance oil can be wicked into bottom plate 110, preparing air freshener 100 to diffuse aroma immediately when placed into use.

In another mode of diffusing aroma, bottom plate 110 includes openings 122 aligned with passageways 210 within scented substrate 202 to form channels 302 for airflow 410 through the device. In some examples, spacers 306 will offset a top surface of scented substrate 202 from a bottom surface of top 112 on housing 108, forming a gap 308 between the two surfaces. As a result, air rising through openings 122, and vapor diffusing within channels 302, will pass into gap 308. In the examples illustrated in the figures, vents 124 at the intersection of top 112 and front 114 and the intersection of top 112 and rear 116 are substantially parallel to gap 308 and substantially orthogonal to channels 302, allowing airflow 410 with fragrance vapor to exit air freshener 100 through vents 124. While forced air may accelerate the dispersion of fragrance, air freshener 100 does not require the presence of forced air and may improve the aroma of an environment when positioned away from typical convection, such as on a visor of vehicle or similar location.

Those of ordinary skill in the field will appreciate that the principles of this disclosure are not limited to the specific examples discussed or illustrated in the figures. For example, while air freshener 100 is illustrated and discussed in the context of placement with clip 102 on visor 104, the device may be attached to any suitable structure and used in any location. In addition, the principles disclosed are not limited to implementation in an interior of a vehicle. Any space deriving benefit from air fragrance could benefit from the examples and techniques disclosed and claimed. Moreover, while the present disclosure addresses an air freshener substantially in the shape of a rectangular prism and resembling a garage-door opener, the shape or form of air freshener 100 containing bottom plate 110 and scented substrate 202 is not limiting.

Unless explicitly excluded, the use of the singular to describe a component, structure, or operation does not exclude the use of plural such components, structures, or operations or their equivalents. As used herein, the word “or” refers to any possible permutation of a set of items. For example, the phrase “A, B, or C” refers to at least one of A, B, C, or any combination thereof, such as any of: A; B; C; A and B; A and C; B and C; A, B, and C; or multiple of any item such as A and A; B, B, and C; A, A, B, C, and C; etc. Also, the use of terms indicating a particular orientation (e.g., “top,” “bottom,” “front,” “rear,” “side,” etc.) is for convenience of description and does not require any particular orientation of the item described.

Terms of approximation are meant to include ranges of values that do not change the function or result of the disclosed structure or process. For instance, the term “about” generally refers to a range of numeric values that one of skill in the art would consider equivalent to the recited numeric value or having the same function or result. Similarly, the antecedent “substantially” means largely, but not wholly, the same form, manner or degree, and the particular element will have a range of configurations as a person of ordinary skill in the art would consider as having the same function or result. As an example, “substantially parallel” need not be exactly 180 degrees but may also encompass slight variations of a few degrees based on the context.

While aspects of the present disclosure have been particularly shown and described with reference to the embodiments above, it will be understood by those skilled in the art that various additional embodiments may be contemplated by the modification of the disclosed systems and methods without departing from the spirit and scope of what is disclosed. Such embodiments should be understood to fall within the scope of the present disclosure as determined based upon the claims and any equivalents thereof.

What is claimed is:

1. An air freshener, comprising:
a fragrant body containing fragrance oil, the fragrant body having at least an inner portion and an outer surface, the outer surface comprising a fibrous material;
channels of open space passing through the fragrant body from the inner surface to the outer surface; and
a housing configured to encase the inner portion of the fragrant body,
wherein the housing and the outer surface of the fragrant body form an exterior of the air freshener.

2. The air freshener of claim 1, wherein the outer surface of the fragrant body is located at a lower portion of the air freshener, the housing further comprising:
one or more vents located at an upper portion of the air freshener, the one or more vents being in communication with the channels.

3. The air freshener of claim 2, wherein the housing is a partial rectangular prism having at least a top, a front, and a rear, a first of the one or more vents being located at an intersection of the top and the front.

4. The air freshener of claim 3, wherein the channels are oblong in shape and extend from the lower portion of the air freshener to the top of the housing.

5. The air freshener of claim 4, wherein a second of the one or more vents is located at an intersection of the top and the rear.

6. The air freshener of claim 4, wherein the fragrant body comprises the fibrous material in contact with a scented substrate, the scented substrate being a polymer compounded with the fragrance oil.

7. The air freshener of claim 6, wherein the scented substrate is polyvinylchloride (PVC) or ethylene-vinyl acetate (EVA).

8. The air freshener of claim 1, wherein, prior to installation, the fragrant body is contained in a sealed package.

9. An air freshener, comprising:
a scented substrate loaded with an oil-based fragrance;
passageways for airflow formed through the scented substrate;
a housing configured to encase the scented substrate, the housing including one or more vents positioned orthogonal to the passageways; and
a gap between the scented substrate and the housing, the gap providing a path for the airflow from one or more of the passageways to the one or more vents.

10. The air freshener of claim 9, further comprising a fibrous material in contact with the scented substrate, the fibrous material and the housing forming an external body of the air freshener.

11. The air freshener of claim 10, wherein the fibrous material is a lignin-based substance.

12. The air freshener of claim 11, wherein the fibrous material is beechwood.

13. The air freshener of claim 10, wherein the fibrous material is at a bottom of the air freshener and includes openings for the airflow, the openings being in registration with the passageways to form channels for the airflow through the air freshener.

14. The air freshener of claim 9, wherein the housing is a partial rectangular prism having at least a top, a front, and a rear, the one or more vents being located at an intersection of the top and the front and at the intersection of the top and the rear.

15. The air freshener of claim 14, further comprising one or more separators positioned between the scented substrate and the top of the housing, the one or more separators forming the gap.

16. The air freshener of claim 9, further comprising a clip attached to the housing.

17. A diffuser, comprising:
a plate of fibrous material;
openings for airflow formed through the fibrous material;
a scented substrate loaded with an oil-based fragrance, the scented substrate being in contact with the plate of fibrous material; and
passageways for the airflow formed through the scented substrate,
wherein the openings are aligned with the passageways to form channels for the airflow through the diffuser.

18. The diffuser of claim 17, further comprising a housing, wherein the housing and the plate encase the scented substrate.

19. The diffuser of claim 18, wherein the housing includes one or more vents at an opposite end of the diffuser from the openings, the one or more vents being orthogonal to the channels.

20. The diffuser of claim 17, further comprising a sealed package enclosing the diffuser to prevent dispersion of the oil-based fragrance.
